(19) United States
(12) Patent Application Publication
(10) Pub. No.: US 2024/0123144 A1
(43) Pub. Date: Apr. 18, 2024

(54) APPARATUSES, SYSTEMS, AND METHODS FOR HEMODYNAMIC MONITORING

(71) Applicant: HONEYWELL INTERNATIONAL INC., Charlotte, NC (US)

(72) Inventor: Kuna KISHORE, Charlotte, NC (US)

(21) Appl. No.: 18/477,681

(22) Filed: Sep. 29, 2023

(30) Foreign Application Priority Data
Oct. 13, 2022 (IN) 202211058513

(51) Int. Cl. A61M 5/168

(52) U.S. Cl. CPC A61M 5/16854; A61M 2230/30

(57) ABSTRACT

Apparatus, systems, and methods for hemodynamic monitoring are provided. In some embodiments, the hemodynamic monitoring includes use of an IV fluid administered through a blood pressure transducer. The blood pressure transducer generates an electrical signal that is transmitted to a hemodynamic monitor to generate a blood pressure measurement. The blood pressure transducer may comprise a first chamber to receive and transmit an IV fluid, a second chamber connected to a reference tube and filed with reference fluid, and a pressure sensor configured to generate the electrical signal the blood pressure measurement is generated from.

### CROSS-REFERENCE TO RELATED APPLICATIONS

This application claims priority pursuant to 35 U.S.C. 119(a) to Indian Application No. 202211058513, filed Oct. 13, 2022, which application is incorporated herein by reference in its entirety.

### TECHNOLOGICAL FIELD

Example embodiments of the present invention relate generally to hemodynamic monitoring, and more particularly to a blood pressure transducer providing pressure correction during hemodynamic monitoring.

### BRIEF SUMMARY

Various embodiments described herein relate to apparatuses, systems, and methods for hemodynamic monitoring.

In accordance with some embodiments of the present disclosure, a blood pressure transducer is provided. The transducer comprises: a first chamber and a second chamber, wherein the first chamber and the second chamber share a first chamber wall that separates the first chamber and the second chamber; a IV fluid output connector connected to the first chamber; a IV fluid input connector connected the first chamber, wherein the first chamber is configured for an IV fluid to flow from the IV fluid input connector to the IV fluid output connector; an opening in the shared first chamber wall; a differential pressure sensor in the second chamber configured to generate an electrical signal, wherein the differential pressure sensor is configured to be in fluidic communication with an IV fluid in the first chamber via the opening in the first chamber wall, wherein the differential pressure sensor is in fluidic communication with a reference fluid in the second chamber, and wherein the electrical signal is configured to be generated based at least on a difference in pressure between an IV fluid in the first chamber and the reference fluid in the second chamber; and a reference tube with a first end and a second end, wherein the first end of the reference tube is fluidically connected to the second chamber, wherein the second end of the reference tube is connected to a membrane configured to be attached to a patient, and wherein the second chamber and the reference tube are filled with the reference fluid.

In accordance with some embodiments of the present disclosure, a hemodynamic monitoring system is provided. The hemodynamic monitoring system comprising: a hemodynamic monitor electrically connected to a differential pressure sensor of a transducer; an IV fluid bag connected to an IV fluid input connector of the transducer; and a patient IV fluid tube connected to the IV fluid output connector of the transducer. In some embodiments, the blood pressure transducer comprises: a first chamber and a second chamber, wherein the first chamber and the second chamber share a first chamber wall that separates the first chamber and the second chamber; a IV fluid output connector connected to the first chamber; a IV fluid input connector connected the first chamber, wherein the first chamber is configured for an IV fluid to flow from the IV fluid input connector to the IV fluid output connector; an opening in the shared first chamber wall; a differential pressure sensor in the second chamber configured to generate an electrical signal, wherein the differential pressure sensor is configured to be in fluidic communication with an IV fluid in the first chamber via the opening in the first chamber wall, wherein the differential pressure sensor is in fluidic communication with a reference fluid in the second chamber, and wherein the electrical signal is configured to be generated based at least on a difference in pressure between an IV fluid in the first chamber and the reference fluid in the second chamber; and a reference tube with a first end and a second end, wherein the first end of the reference tube is fluidically connected to the second chamber, wherein the second end of the reference tube is connected to a membrane configured to be attached to a patient, and wherein the second chamber and the reference tube are filled with the reference fluid.

In accordance with some embodiments of the present disclosure, a method for hemodynamic monitoring is provided. The method for hemodynamic monitoring comprises: electrically connecting a hemodynamic monitor to a differential pressure sensor of a transducer, wherein the transducer comprises: a first chamber and a second chamber, wherein the first chamber and the second chamber share a first chamber wall that separates the first chamber and the second chamber; a IV fluid output connector fluidically connected to the first chamber; a IV fluid input connector connected the first chamber, wherein the first chamber is configured for an IV fluid to flow from the IV fluid input connector to the IV fluid output connector; an opening in the shared first chamber wall; a differential pressure sensor in the second chamber configured to generate an electrical signal, wherein the differential pressure sensor is configured to be in fluidic communication with an IV fluid in the first chamber via the opening in the first chamber wall, wherein the differential pressure sensor is in fluidic communication with a reference fluid in the second chamber, and wherein the electrical signal is configured to be generated based at least on a difference in pressure between an IV fluid in the first chamber and the reference fluid in the second chamber; and a reference tube with a first end and a second end, wherein the first end of the reference tube is fluidically connected to the second chamber, wherein the second end of the reference tube is connected to a membrane configured to be attached to a patient, and wherein the second chamber and the reference tube are filled with the reference fluid. The method for hemodynamic monitoring further comprises: connecting an IV fluid bag to the IV fluid input connector, wherein the IV fluid bag contains an IV fluid; connecting a first end of a patient IV fluid tube to the IV fluid output connector; connecting a second end of the patient IV fluid tube to a patient; connecting the second end of the reference tube to the patient; providing the IV fluid to the patient from the IV fluid bag via the transducer and the patient IV fluid tube; generating the electrical signal with the differential pressure sensor; transmitting the electrical signal to the hemodynamic monitor; and measuring a blood pressure of the patient by the hemodynamic monitor based on at least the electrical signal.

In some embodiments, the electrical signal is an analog signal.

In some embodiments, the electrical signal is a digital signal.

In some embodiments, the differential pressure sensor comprises an external membrane, wherein the external membrane is in fluid contact with IV fluid.

In some embodiments, the reference fluid and the IV fluid are the same fluid.

In some embodiments, the reference fluid and the IV fluid are not the same fluid.

In some embodiments, the first chamber and the second chamber are located inside a transducer housing, an exterior wall of the transducer housing includes an identifier configured to be optically recognized, and the identifier is associated with the reference fluid.

The foregoing brief summary is provided merely for purposes of summarizing some example embodiments illustrating some aspects of the present disclosure. Accordingly, it will be appreciated that the above-described embodiments are merely examples and should not be construed to narrow the scope of the present disclosure in any way. It will be appreciated that the scope of the present disclosure encompasses many potential embodiments in addition to those summarized herein, some of which will be described in further detail below.

### BRIEF SUMMARY OF THE DRAWINGS

Having thus described certain example embodiments of the present disclosure in general terms, reference will now be made to the accompanying drawings, which are not necessarily drawn to scale, and wherein:

FIG. 1A illustrates a conventional hemodynamic monitoring system utilizing a conventional blood pressure transducer;

FIG. 1B illustrates a block diagram of a conventional blood pressure transducer;

FIG. 1C illustrates a graph demonstrating errors generated in the conventional hemodynamic monitoring system in a convention hemodynamic monitoring system;

FIG. 2 illustrates a block diagram of a blood pressure transducer in accordance with one or more embodiments of the present invention;

FIG. 3 illustrates a hemodynamic monitoring system utilizing a blood pressure transducer in accordance with one or more embodiments of the present invention;

FIG. 4 illustrates an example block diagram of a hemodynamic monitor in accordance with one or more embodiments of the present invention; and

FIG. 5 illustrates a flowchart according to an example method for hemodynamic monitoring with one or more embodiments of the present disclosure.

### DETAILED DESCRIPTION

Some embodiments of the present disclosure will now be described more fully herein with reference to the accompanying drawings, in which some, but not all, embodiments of the disclosure are illustrated. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout.

As used herein, the term “comprising” means including but not limited to and should be interpreted in the manner it is typically used in the patent context. Use of broader terms such as comprises, includes, and having should be understood to provide support for narrower terms such as consisting of, consisting essentially of, and comprised substantially of.

The phrases “in one embodiment,” “according to one embodiment,” “in some embodiments,” and the like generally mean that the particular feature, structure, or characteristic following the phrase may be included in at least one embodiment of the present disclosure and may be included in more than one embodiment of the present disclosure (importantly, such phrases do not necessarily refer to the same embodiment).

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments or it may be excluded.

The use of the term “circuitry” as used herein with respect to components of a system or an apparatus should be understood to include particular hardware configured to perform the functions associated with the particular circuitry as described herein. The term “circuitry” should be understood broadly to include hardware and, in some embodiments, software for configuring the hardware. For example, in some embodiments, “circuitry” may include processing circuitry, communication circuitry, input/output circuitry, and the like. In some embodiments, other elements may provide or supplement the functionality of particular circuitry.

The term “electronically coupled,” “electronically coupling,” “electronically couple,” “in communication with,” or “in electronic communication with,” in the present disclosure refers to two or more elements, modules, circuitry, or components being connected through wired means and/or wireless means, such that signals, electrical voltage/current, data and/or information may be transmitted to and/or received from these elements or components.

### Overview

The present invention is related to hemodynamic monitoring, including improved apparatuses, systems, and methods for hemodynamic monitoring.

In conventional hemodynamic monitoring systems, a conventional blood pressure transducer that induces hemodynamic pressure monitoring errors when a patient moves. Hemodynamic monitoring utilizes intravenous (IV) fluids connected to a patient to monitor the patient's blood pressure. The IV fluid is connected to patient, specifically to a blood vessel (e.g., vein, artery, etc.) of a patient via an IV tube's cannula. This allows for blood pressure wave measurement using the IV fluid as a pressure propagating medium. As a patient's heart beat creates pressure in the patient, the pressure is hydraulically communicated through the IV fluid to the hemodynamic pressure monitoring system. Conventional hemodynamic monitoring systems utilize a hemodynamic pressure transducer that introduces measurement errors due to calibration issues. Specifically, patient movement at the distal end of an IV's cannula, particularly in relation to the proximal end of the IV's cannula connected to a conventional blood pressure transducer housing and its pressure sensor, induces a hydrostatic pressure error associated with the patient's movement. This hydrostatic pressure error shifts the baseline of a blood pressure wave that is being measured. The shift in the baseline causes a conventional hemodynamic monitoring system to generate false alarms.

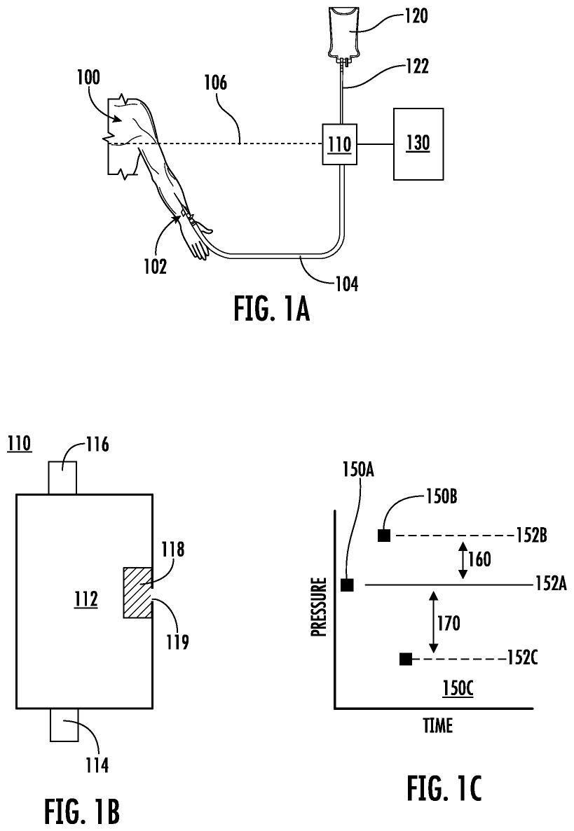

FIG. 1A illustrates a conventional hemodynamic monitoring system utilizing a conventional blood pressure transducer. This conventional hemodynamic monitoring system connects to a patient 100 via an IV supply tube 104, such as with a catheter 102. The IV supply tube 104 is connected to a conventional blood pressure transducer 110, which is fluidically connected to an IV supply bag 120 via a patient IV fluid tube 122. The IV supply bag 120 is pressurized, which provides a positive pressure to assist the IV fluid being provided to the patient. The conventional blood pressure transducer 110 is also electrically connected with a hemodynamic monitor 130.

The IV supply tube 104 is connected to a patient 100, such as at a patient's arm via a catheter 102. To make hemodynamic measurements, the conventional hemodynamic monitoring system is calibrated such that the conventional blood pressure transducer 110 is aligned with the phlebostatic axis 106 of the patient 100, which is an anatomical point corresponding to the right atrium. However, such a calibration is susceptible to error whenever the patient moves, which causes the connection (e.g., 102, 104) to the patient 100 to move. Such movement may be as small as the patient 100 moving their arm (e.g., raising an arm, lowering an arm, an arm falling out of bed, etc.), sitting up in bed, or laying down in bed. Each of these movements are common adjustments a patient 100 may make tens or hundreds of times a day, often without realizing that an error is being introduced into the conventional hemodynamic monitoring system.

The calibration (and recalibration) of the conventional hemodynamic monitoring system is burdensome on trained medical professionals. The calibration (and recalibration) includes aligning the conventional blood pressure transducer 110 with the phlebostatic axis 106 and entering a calibration setting in the hemodynamic monitor 130 to zero the conventional blood pressure transducer 110. For example, to calibrate a conventional hemodynamic monitoring system, a trained professional (e.g., doctor, nurse, technician, etc.) may use a level (a.k.a. carpenter level, standard level, etc.) or equivalent instrument to set the level of the conventional blood pressure transducer 110 with a patient's phlebostatic axis 106. Zero or zeroing calibrates the hemodynamic monitor 130 to a pressure measurement associated with the atmospheric pressure at that level. Thus patient movement above or below the level induces error.

FIG. 1B illustrates a block diagram of a conventional blood pressure transducer 110. The conventional blood pressure transducer 110 includes a single chamber 112 with an IV fluid output connector 114 and an IV fluid input connector 116 for connection to two IV fluid lines: the IV supply tube 104 and the patient IV tube 122. IV fluid is supplied to the conventional blood pressure transducer 110 from an IV fluid bag 120 via the IV fluid input connector 116. The IV fluid flows through the chamber 112 of the conventional blood pressure transducer 110 and to a patient 100 via the IV fluid output connector 114 and the IV supply tube 104. Once connected to a patient 100, the IV fluid propagates the patient's heartbeat to a pressure sensor 118 included in the chamber 112 of the conventional blood pressure transducer 110.

The pressure sensor 118 of the conventional blood pressure transducer 110 is located in the chamber 112. A first side of the pressure sensor 118 is exposed to the IV fluid flowing through the chamber 112. A second side of the pressure sensor 118 may be exposed to atmospheric pressure, such as via an opening 119 in the chamber 112. The conventional blood pressure transducer 110 is connected via an electrical connection to a hemodynamic monitor 130 that turns the electrical signal into a pressure reading.

FIG. 1C illustrates a graph demonstrating errors generated by the conventional blood pressure transducer 110 in a convention hemodynamic monitoring system. The graph illustrates pressure and time, and it will be appreciated that a pressure wave is not illustrated. Instead the graph illustrates the errors from hydrostatic pressure error offsets that will be introduced by conventional blood pressure transducer 110 due to patient movement. FIG. 1C illustrates a patient position 150 at three different locations as a patient may move from a time when the conventional hemodynamic monitoring system was calibrated. In FIG. 1C, the conventional hemodynamic monitoring system was calibrated with a patient's in a first position at 150A for a zeroing of the conventional hemodynamic monitoring system at level 152A to create a baseline. The patient 100 moves, such as sitting up in bed that induces a pressure that causes the pressure to rise, which may raise the patient for a first adjusted position 150B, which corresponds to a level 152B, which introduces an error 160 representing a hydrostatic pressure error. The patient 100 may move again, such as laying down in bed that induces a pressure drop causing the pressure to fall, which may lower the patient for a second adjusted position 150C, which corresponds to a level 152C, which introduces an error 170 representing a hydrostatic pressure error from the level 150. Both errors 160 and 170 are from an initial level 152A. Such movements from 150A to 150B and/or 150C may have a patient 100 moving as little as 3 centimeters, but this may result in a hydrostatic pressure error at or near 2 mmHg, which may be outside of the acceptable limit and generate false alarms. Thus the errors 160 and 170 may generate erroneous reading and generate alarms in the hemodynamic monitor 130. Such alarms due to movement require that a trained professional use the hemodynamic monitor 130 to clear the alarm and recalibrate the hemodynamic monitor 130 due to the change in position of the patient 100.

In various embodiments, alarms may also be related to maintenance or a potential failure of one or more components of the transducer 200. For example, blood pressure measurements typically include waves, and a measurements that has a consistently low, high, or non-changing level may generate an alarm to indicate that maintenance or a potential failure of a transducer 200.

Embodiments of the present invention described herein include a blood pressure transducer configured to address the hydrostatic pressure error introduced in conventional hemodynamic monitoring systems, particularly the hydrostatic pressure difference due to one or more changes in relative position of a transducer with a patient's phlebostatic axis. In particular, the induced hydrostatic pressure error introduced by the patient movement is offset with the transducer described herein by capturing the same patient movement as a correcting pressure. Embodiments of the blood pressure transducer of the present invention include a differential pressure sensor, where a reference port of a blood pressure transducer allows for the measurement of an induced hydrostatic pressure error so that the differential pressure sensor may compensate for the hydrostatic pressure error.

### Exemplary Apparatus and System

Embodiments of the present invention herein include a blood pressure transducer that provides for automatic correction of the induced hydrostatic pressure error change due to patient movement.

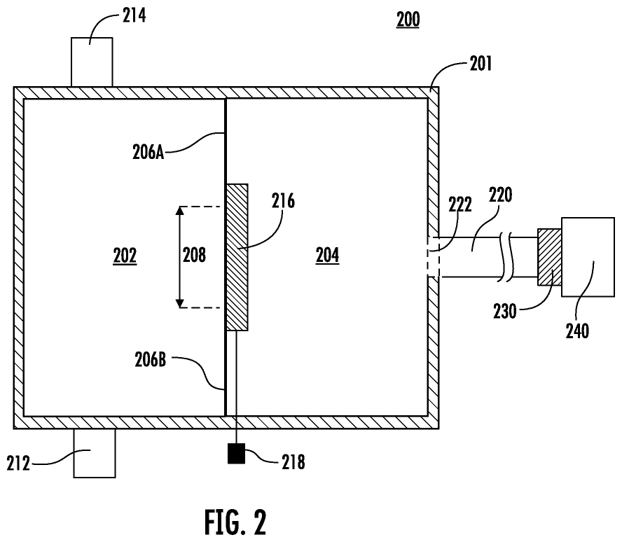

FIG. 2 illustrates a block diagram of a blood pressure transducer 200 in accordance with one or more embodiments of the present invention. In various embodiments, the blood pressure transducer 200 may include a transducer housing 201 containing at least a first chamber 202 and a second chamber 204. In various embodiments, the transducer housing 201, the first chamber 202, and the second chamber 204 are sealed so as to not allow a fluid leak as described herein. In various embodiments, the transducer housing 201 may be made of biocompatible materials, such as a biocompatible plastic, acrylonitrile butadiene styrene, clear polycarbonates, PVC, and the like.

The first chamber 202 and the second chamber 204 may share a chamber wall 206. The chamber wall 206 may include one or more openings 208. The blood pressure transducer 200 may include a pressure sensor 216, a first side of which covers the one or more openings 208 such that a fluid in the first chamber 202 is separated from a fluid in the second chamber 204. In various embodiments, the pressure sensor 216 may be a differential pressure sensor in fluid communication with the IV fluid of the first chamber 202 and also in fluid communication with the reference fluid of the second chamber 204. The pressure sensor 216 may generate an electrical signal based on the pressure from the IV fluid in the first chamber 202 and the pressure from the fluid in the second chamber 204, particularly the difference between the IV fluid in the first chamber 202 and the pressure from the fluid in the second chamber 204. The electrical signal may be output to one or more electrical connectors 218. In various embodiments, the one or more electrical connectors 218 may be connected to a hemodynamic monitor 330.

In various embodiments, the pressure sensor 216 may be a micro-electromechanical system (MEMS) sensor that convert pressure to an electrical signal. The pressure may be a differential pressure, such as a pressure from an IV fluid of chamber 202 and a reference fluid of chamber 204. In various embodiments, the pressure sensor 216 may provide an electrical signal output after receiving an electrical input. For example, the pressure sensor 216 may include one or more resistors that change in resistance as one or more pressures are applied to the pressure sensor 216, and this may generate a waveform output. Thus the pressure sensor 216 may receive a voltage signal, change the voltage signal via the resistors, and output the changed voltage signal, which may be measured by a hemodynamic monitor 330. In various embodiments the output electrical signal may be an analog signal. In various embodiments the output electrical signal may be an digital signal. In various embodiments, the pressure sensor 216 may include a pressor and a memory, such as described herein, that may generate the digital signal. In various embodiments, blood pressure transducer 200 may include a pressor and a memory located in the blood pressure transducer 200 but outside the pressure sensor 216 that may generate the digital signal. In various embodiments, a processor may convert an analog signal into a digital signal, which may also include instructions and/or a program for the conversion being stored in memory and executed by the processor.

In various embodiments, the pressure sensor 216 may include an external layer encompassing a portion or all of the pressure sensor 216. The external layer may be a silicon layer an external membrane (e.g., a PTFE membrane, silicone gel, etc.). For example, a first external layer may encompass some or all of the portions of the pressure sensor 216 in contact and/or fluidically connected to an IV fluid in the first chamber 202, and a second external layer may encompass some or all of the portions of the pressure sensor 216 in contact and/or fluidically connected to a reference fluid in the second chamber 204.

The first chamber 202 may include or be connected to a IV fluid output connector 212 and a IV fluid input connector 214. Each of the IV fluid output connector 212 and the IV fluid input connector 214 may include and/or be comprised of a connector and/or a port, such as but not limited to a luer connector or the like. The connector may be configured to connect to IV tubing. In various embodiments one of or each of the connectors may additionally be configured to control the flow either to or from IV tubing, such as with one or more valves. For example, a connector may include a two way valve, a three way value, a four way valve, or the like.

The second chamber 204 may include or be connected to a reference tubing 220. The connection may be integrated into the second chamber 204 or may be connected to the second chamber 204 via a reference port 222. Reference port 222 is illustrated in FIG. 2 as an opening, but it will be appreciated that reference port 222 may be a connector or port, similar to connectors IV fluid output connector 212 and/or IV fluid input connector 214. The reference tubing 220 may be of a same length as an IV supply tube 104 to be used with the blood pressure transducer 200. The reference tubing 220 may be terminated at a termination 230. The termination 230 may include an fluid tight but air permeable membrane (e.g., a PTFE membrane, Gore-Tex® membrane, or the like). The termination 230 may keep fluid in the blood pressure transducer 200 such that the second chamber 204 and reference tubing 220 are fluid filled. In various embodiments, this fluid in the blood pressure transducer 200 may be filled during manufacturing such that there is no compressible air or gas in the second chamber 204. In use, the air permeable termination 230 allows for the atmospheric pressure at the termination 230 to be applied through the fluid to the pressure sensor 216, which generates an offsetting pressure to the hydrostatic pressure error, sometimes referred to as a corrective pressure.

While FIG. 2 illustrates the reference port 222 on a side of the transducer 200, it will be appreciated that the reference port 200 may be located anywhere on the transducer 200 that allows for a fluidic connection to the reference tube 220.

In various embodiments, the termination 230 may be covered by a removable cap 240. The removeable cap 240 may be used in packaging and during transportation of a blood pressure transducer 200 to protect the termination 230 and prevent damage. During use, the removable cap 240 may be removed and the termination 230 may be connected to a patient with a connector (e.g., medical tape, a clip, or the like). In various embodiments, such a connection to the patient does not physiologically interface with the patient. The termination 230, when placed at or near the catheter 102 providing IV fluids to the patient 100, provides automatic correction of induced hydrostatic pressure error change due to patient movement. In various embodiments, with the termination 230 being at or near the catheter 102, when a patient 100 moves then the termination 230 moves, which causes the atmospheric pressure applied on the liquid in the reference tubing 220 to have a different atmospheric pressure. Thus the blood pressure transducer 200 mechanically provides an offsetting pressure through the termination 230 that corrects pressure captured from the patient movement to eliminate hydrostatic pressure error in conventional hemodynamic monitoring systems.

A cap 240 may be provided over termination 230, such as in packaging and during transport. The cap 240 may be kept on the tube during transit and storage and before use.

In various embodiments, the IV fluid and the reference fluid may be the same fluid (e.g., water, saline, etc.). In various embodiments, the IV fluid and the reference fluid may not be the same fluid (e.g., different fluids may be two of saline, water, blood, mineral oil, silicone oil, etc.). In other words, the IV fluid and the reference fluid may be different fluids. In various embodiments, when a different fluid is used, an identifier on the transducer 200 may identify the reference fluid, which may be used to calibrate a hemodynamic monitor 330. This calibration, including an identification and entering of a reference fluid, may allow for the hemodynamic monitor to compensate or adjust a blood pressure measurement based on the reference fluid, particularly if the reference fluid may have a different density than the IV fluid. In such embodiments, the IV fluid being administered would also be input into the hemodynamic monitor 330, which may be based on one or more identifiers associated with the IV fluid, such as on the IV supply bag 120. Entering the information in the identifier may be via an input/output circuitry or through communications circuitry of the hemodynamic monitor 330, which are described herein.

The differential pressure sensor 216 accounts for this correcting pressure by adding or subtracting the induced hydrostatic pressure error when accounting for the difference between the IV fluid and the prefilled fluid. Any patient 100 movement causes an induced hydrostatic pressure error in the fluid in the second chamber 204 that is measured the differential pressure sensor 216 over and above actual blood pressure wave transmitted by the IV fluid in the first chamber 202. Thus the net static pressure seen by differential pressure sensor 216 is zero, which offsets the error due to patient 100 movement.

In various embodiments, use of the blood pressure transducer 200 also eliminates manual zero-point setting of the pressure sensor 216 at the hemodynamic monitor 330 as the differential pressure sensor 216 is zeroed with the two hydrostatic pressures from both the IV supply tube 104 and the reference tubing 220. This improvements patient care, including by generating time savings for a medical professional when preparing the hemodynamic monitoring system. It may also allow for the blood pressure transducer to be located anywhere on a stand or rack, which may be holding a number of IV fluids and/or monitors.

The blood pressure transducer 200 of the present invention may be included in a hemodynamic catheter kit or set. Such a hemodynamic catheter kit or set may include all of the components needed for a medical professional to introduce an IV fluid into a patient except for the IV fluid in an IV fluid supply and the hemodynamic monitor 330. For example, a hemodynamic catheter kit or set may include a hemodynamic pressure transducer 200, IV supply tube 104, patient IV fluid tube 122, catheter 102, guide needles, IV tubing connections and/or valves, and the like. The hemodynamic catheter kit may come packaged in a sealed package, which may be sealed after the components of the hemodynamic catheter kit have been sterilized. In this manner the hemodynamic catheter kit may be taken to a patient 100, such as at a patient's bedside, and opened for immediate usage by a medical professional. In various embodiments, the contents of a hemodynamic catheter kit or set may be disposable.

Additionally, the hemodynamic catheter kit packaging may include one or more package identifiers, such as serial number, expiration date, barcode, QR code, or the like. In various embodiments, one or more of the package identifiers may be utilized with optical recognition to identifier the contents of the hemodynamic catheter kit, including the identification of the prefilled fluid of the blood pressure transducer 200. The hemodynamic monitor 330 may be in communication with an optical reader that may read and optically recognize the identifier. In various embodiments, the hemodynamic monitor 330 may then utilize the identification of the prefilled fluid to calibrate the hemodynamic monitor 330. In various embodiments, the blood pressure transducer 200 may also, or alternatively, include an identifier containing the same or similar information.

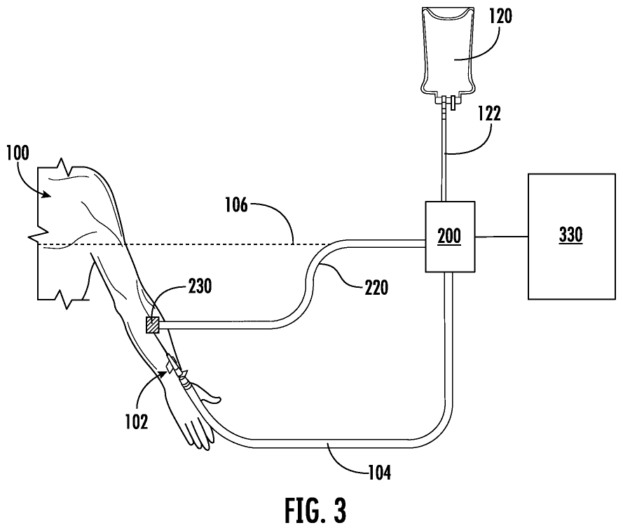

FIG. 3 illustrates a hemodynamic monitoring system utilizing a blood pressure transducer 200 in accordance with one or more embodiments of the present invention. This hemodynamic monitoring system includes a blood pressure transducer 200 (including the reference tube 220, etc.), a hemodynamic monitor 330, an IV supply bag 120, patient IV fluid tube 122, IV supply tube 104, and catheter 102.

In various embodiments, the reference tubing 220 may be run along the IV supply tube 104. In various embodiments, the reference tubing 220 or the IV supply tube 104 may include one or more tube connectors (e.g., clamps, straps, or the like) that may connect the reference tubing 220 and the IV supply tube 104 so that they are physically connected. In various embodiments, the reference tubing 220 and the IV supply tube 104 may be physically connected together except for a length on each end of the reference tubing 220 or the IV supply tube 104, with such length being designed to allow for connections to where each of the reference tubing 220 and the IV supply tube 104 respectively terminates.

In various embodiments, in use the IV fluid input connector 214 of the blood pressure transducer 200 is opened to IV fluid path. The IV fluid has filled a transducer flow path, including the first chamber 202 and IV supply tube 104, and the IV supply tube 104 is pressurized from the IV supply bag 120, which allows for the IV fluid to serve as a pressure propagating medium to communicate a blood pressure wave of a patient 100. This blood pressure wave may be measured over and above any static pressure present in the hemodynamic monitoring system.

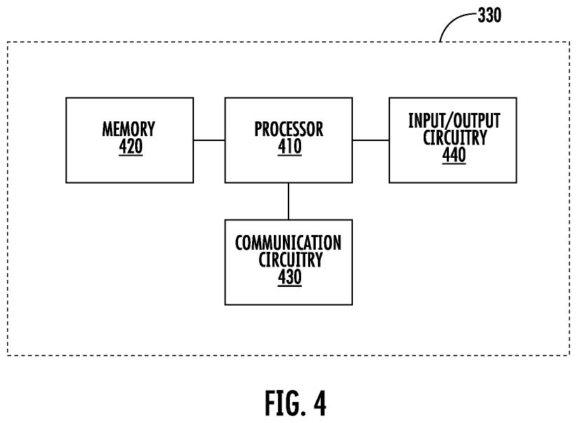

FIG. 4 illustrates an example block diagram of a hemodynamic monitor 330 in accordance with one or more embodiments of the present invention. As illustrated, the hemodynamic monitor 330 may comprise a processor 410, memory 420, communication circuitry 430, input/output circuitry 440, and/or other components configured to perform various operations, procedures, functions, or the like described herein.

The processor 410, although illustrated as a single processor 410, may be comprised of a plurality of components and/or processor circuitry. The processor 410 may be implemented as, for example, various devices comprising one or a plurality of microprocessors with accompanying digital signal processors; one or a plurality of processors without accompanying digital signal processors; one or a plurality of coprocessors; one or a plurality of multi-core processors; processing circuits; one or a plurality of computers; and various other processing elements (including integrated circuits, such as ASICs or FPGAs, or a certain combination thereof). In various embodiments, the processor 410 may be configured to execute instructions and/or programs stored in the memory 420 or otherwise accessible by the processor 410. When executed by the processor 410, these instructions and/or programs may enable the execution of one or a plurality of the operations and/or functions described herein. Regardless of whether it is configured by hardware, firmware/software methods, or a combination thereof, the processor 410 may comprise entities capable of executing operations and/or functions according to the embodiments of the present invention when correspondingly configured. Therefore, for example, when the processor 410 is implemented as an ASIC, an FPGA, or the like, the processor 410 may comprise specially configured hardware for implementing operations and/or functions described herein.

The memory 420 may comprise, for example, a volatile memory, a non-volatile memory, or a certain combination thereof. Although illustrated as a single memory 420, the memory 420 may comprise a plurality of memory components. In various embodiments, the memory 420 may comprise, for example, a hard disk drive, a random access memory, a cache memory, a flash memory, an optical disk, a circuit configured to store information, or a combination thereof. The memory 420 may be configured to store data, information, application programs, instructions, etc., so that the hemodynamic monitor 330 may execute various operations and/or functions according to the embodiments of the present disclosure.

The communication circuitry 430 may be implemented as any apparatus included in a circuit, hardware, computer program product, or a combination thereof, which is configured to receive and/or transmit data from/to another component or apparatus. The computer program product may comprise computer-readable program instructions stored on a computer-readable medium (e.g., memory 420) and executed by a hemodynamic monitor 330 (e.g., processor 410). In various embodiments, the communication circuitry 430 (as with other components discussed herein) may be at least partially implemented as part of the processor 410 or otherwise controlled by the processor 410. The communication circuitry 430 may communicate with the processor 410, the memory 420, the input/output circuitry 440, and/or any other component, for example, through a bus. Such a bus may connect to the processor 410, and it may also connect to one or more other components of the hemodynamic monitor 330. The communication circuitry 430 may be comprised of, for example, antennas, transmitters, receivers, transceivers, network interface cards and/or supporting hardware and/or firmware/software, and may be used for establishing communication with another system, apparatus, and/or component. The communication circuitry 430 may be configured to receive and/or transmit any data that may be stored by, for example, the memory 420 by using any protocol that can be used for communication between components and/or apparatuses. In various embodiments, the communication circuitry 430 may transform or package data to be transmitted and/or transform or unpackage data received, such as from a first communication protocol to a second communication protocol or from a first data type to a second data type.

The input/output circuitry 440 may communicate with the processor 410 to receive instructions input by a user and/or to provide audible, visual, mechanical, or other outputs to a user. The input/output circuitry 440 may comprise supporting devices, such as a keyboard, a mouse, a user interface, a display, a touch screen display, lights (e.g., warning lights), indicators, speakers, and/or other input/output mechanisms. In various embodiments, aspects of the input/output circuitry 440 may be implemented on a device used by the user to communicate with the hemodynamic monitor 330. The input/output circuitry 440 may communicate with the memory 420, the communication circuitry 430, and/or any other component, for example, through a bus.

A blood pressure transducer 200 may communicate with a hemodynamic monitor 330 via the communication circuitry 430. The pressure sensor 216 may generate an electrical signal that may be transmitted to an electrical connector 218 of the blood pressure transducer 200, which may be connected to the communications circuitry 430 of the hemodynamic monitor 330. In various embodiments, the electrical signal generated and transmitted may be an analog signal. In various embodiments, the electrical signal generated and transmitted may be a digital signal.

The hemodynamic monitor 330, based on the electrical signals received from the blood pressure transducer 200, generates one or more visual displays. For example, a hemodynamic monitor 330 may generate and display a visual display of a patient's blood pressure over time. The blood pressure over time may be displayed as a blood pressure waveform. A blood pressure waveform has certain characteristics that may be interpreted by the medical practitioner to know the status of the patient.

Additionally, and also based on the electrical signals received from the blood pressure transducer 200, the hemodynamic monitor 330 may generate one or more alarms.

Having generally described embodiments in accordance with the present invention, several exemplary operations according to exemplary embodiments will be described.

### Exemplary Operations

In some example embodiments, and according to the operations described herein, the blood pressure transducer 200 may be used as a port of a hemodynamic monitoring system to monitor a patient's blood pressure, including generating alarms. While the following flowchart and related description includes multiple operations, it is readily appreciated that some of the following operations may be omitted, some of the operations may be repeated or iterated, and that additional operations may be included. Additionally, the order of operations should not be interpreted as limiting as the order of these operations may be varied.

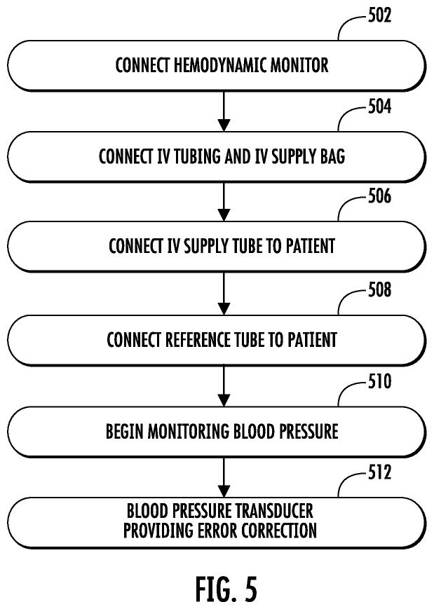

FIG. 5 illustrates a flowchart according to an example method for hemodynamic monitoring with one or more embodiments of the present disclosure.

At operation 502, a hemodynamic monitor 330 may be connected. The hemodynamic monitor 330 may be connected to the blood pressure transducer 200 via one or more electrical connectors 218, which are described herein.

At operation 504, IV supply tube 104, patient IV fluid tube 122, and an IV supply bag 120 are connected. The IV supply tube 104 may be connected to a IV fluid output connector 212 of the blood pressure transducer 200, and patient IV fluid tube 122 may be connected to a IV fluid input connector 214 of the blood pressure transducer 200. The IV supply bag 120 may be connected an end of the patient IV fluid tube 122 not connected to the IV fluid input connector 214. Thus IV fluid may be supplied from the IV supply bag 120 to the blood pressure transducer 200, specifically the first chamber 202, via patient IV fluid tube 122 connecting the IV supply bag 120 to the IV fluid input connector 214 of first chamber 202.

In various embodiments, IV supply tube 104 and patient IV fluid tube 122 may already be connected to a blood pressure transducer 200, such as in a hemodynamic catheter kit. It will be appreciated that if the IV supply tube 104 and patient IV fluid tube 122 is already connected to the blood pressure transducer 200 then portions of operation 502 may be omitted.

Additionally, the blood transducer housing 200 may include one or more flow valves, such as at each of the IV fluid output connector 212 and the IV fluid input connector 214. Such flow valves may be in a closed position or an open position. When connecting the IV supply bag 120 at operation 502, such flow valves may be kept closed or a valve between the IV supply bag 120 and the blood pressure transducer 200 may be opened to allow IV fluid to fill the first chamber.

At operation 506, an IV supply tube 104 is connected to a patient. In various embodiments, the IV supply tube 104 may be terminated at its distal end with a catheter 102 and/or a guide needle. A user (e.g., a medical professional) may use the guide needle to connect the catheter 102 to a patient's blood vessel. The medical professional then may connect the IV supply tube 104 to the patient 100, which will allow the IV fluid of the IV supply bad 120 to be provided to the patient 100. In various embodiments with one or more valves, the valves may have been opened to allow for an IV fluid to be provided.

At operation 508, the reference tubing 220 is connected to the patient. The reference tubing 220 may be connected by its termination 230. The connection may be at or near the location the catheter 102 connects to the patient 100.

At operation 510, blood pressure monitoring begins. The hemodynamic monitoring system 300, with the blood pressure transducer 200 begins generating a pressure signal that is communicated to the hemodynamic monitor 330.

In various embodiments, calibration of the hemodynamic monitor 330 is not required, and the user (e.g., medical professional) may interact with the hemodynamic monitor 330, such as through one or more interfaces of the input/output circuitry 440, to begin monitoring the patient 100's blood pressure. The hemodynamic monitor 330 may generate one or more visualizations based on the electrical signal from the blood pressure transducer 200.

In various embodiments, prior to beginning blood pressure monitoring, a user (e.g., medical professional) may calibrate the hemodynamic monitor 330 to a base level.

At operation 512, error correction is provided by the blood pressure transducer 200. After blood pressure monitoring has begun, a patient 100 may move. The movement, due to the blood pressure transducer 200, particularly the reference tubing 220, may generate a change in atmospheric pressure on both the termination 230 of the reference tubing 220 and the catheter 102. The blood pressure transducer 200, specifically the pressure sensor 216, measures the pressure from the IV fluid and from the reference fluid in the second chamber 204 and, thus, provides error correction, which is incorporated into the signal generated by the pressure sensor 216 that is communicated to the hemodynamic monitor 330.

For example, the blood pressure of a patient 100 may be measured by the pressure sensor 216. The pressure sensor 216 may generate an electrical signal based on the differential pressure between an IV fluid being provided to a patient and the reference fluid. Thus the electrical signal may be based at least on a difference in a pressure from the IV fluid and a pressure from the reference fluid. The electrical signal may be transmitted from the pressure sensor 216 of the transducer 200 to a hemodynamic monitor 330 via the one or more electrical connectors 218. The hemodynamic monitor may generate a blood pressure measurement based at least on the electrical signal. As a patient 100 moves, the blood pressure sensor 216 generates an electrical signal based on the IV fluid and reference fluid, which accounts for and provides error correction in the blood pressure measurements. As blood pressure measurements are taken over time, blood pressure waves may be generated.

Operations and/or functions of the present invention have been described herein, such as in flowcharts. As will be appreciated, computer program instructions may be loaded onto a computer or other programmable apparatus (e.g., hardware) to produce a machine, such that the resulting computer or other programmable apparatus implements the operations and/or functions described in the flowchart blocks herein. These computer program instructions may also be stored in a computer-readable memory that may direct a computer or other programmable apparatus to operate and/or function in a particular manner, such that the instructions stored in the computer-readable memory produce an article of manufacture, the execution of which implements the operations and/or functions described in the flowchart blocks. The computer program instructions may also be loaded onto a computer or other programmable apparatus to cause a series of operations to be performed on the computer or other programmable apparatus to produce a computer-implemented process such that the instructions executed on the computer or other programmable apparatus provide operations for implementing the functions and/or operations specified in the flowchart blocks. The flowchart blocks support combinations of means for performing the specified operations and/or functions and combinations of operations and/or functions for performing the specified operations and/or functions. It will be understood that one or more blocks of the flowcharts, and combinations of blocks in the flowcharts, can be implemented by special purpose hardware-based computer systems which perform the specified operations and/or functions, or combinations of special purpose hardware with computer instructions.

While this specification contains many specific embodiments and implementation details, these should not be construed as limitations on the scope of any disclosures or of what may be claimed, but rather as descriptions of features specific to particular embodiments of particular disclosures. Certain features that are described herein in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

While operations and/or functions are illustrated in the drawings in a particular order, this should not be understood as requiring that such operations and/or functions be performed in the particular order shown or in sequential order, or that all illustrated operations be performed, to achieve desirable results. In certain circumstances, operations and/or functions in alternative ordering may be advantageous. In some cases, the actions recited in the claims may be performed in a different order and still achieve desirable results. Thus, while particular embodiments of the subject matter have been described, other embodiments are within the scope of the following claims.

1. A transducer comprising:
a first chamber and a second chamber, wherein the first chamber and the second chamber share a first chamber wall that separates the first chamber and the second chamber;
a IV fluid output connector connected to the first chamber;
a IV fluid input connector connected the first chamber, wherein the first chamber is configured for an IV fluid to flow from the IV fluid input connector to the IV fluid output connector;
an opening in the shared first chamber wall;
a differential pressure sensor in the second chamber configured to generate an electrical signal, wherein the differential pressure sensor is configured to be in fluidic communication with an IV fluid in the first chamber via the opening in the first chamber wall, wherein the differential pressure sensor is in fluidic communication with a reference fluid in the second chamber, and wherein the electrical signal is configured to be generated based at least on a difference in pressure between an IV fluid in the first chamber and the reference fluid in the second chamber; and
a reference tube with a first end and a second end, wherein the first end of the reference tube is fluidically connected to the second chamber, wherein the second end of the reference tube is connected to a membrane configured to be attached to a patient, and wherein the second chamber and the reference tube are filled with the reference fluid.

2. The transducer of claim 1, wherein the electrical signal is an analog signal.

3. The transducer of claim 1, wherein the electrical signal is a digital signal.

4. The transducer of claim 1, wherein the differential pressure sensor comprises an external membrane, wherein the external membrane is in fluid contact with IV fluid.

5. The transducer of claim 1, wherein the reference fluid and the IV fluid are the same fluid.

6. The transducer of claim 1, wherein the reference fluid and the IV fluid are not the same fluid.

7. The transducer of claim 1, wherein the first chamber and the second chamber are located inside a transducer housing, wherein an exterior wall of the transducer housing includes an identifier configured to be optically recognized, and wherein the identifier is associated with the reference fluid.

8. A system comprising:
a hemodynamic monitor electrically connected to a differential pressure sensor of a transducer;
an IV fluid bag connected to an IV fluid input connector of the transducer;
a patient IV fluid tube connected to the IV fluid output connector of the transducer; and
wherein the transducer comprises:
a first chamber and a second chamber, wherein the first chamber and the second chamber share a first chamber wall that separates the first chamber and the second chamber;
a IV fluid output connector connected to the first chamber;
a IV fluid input connector connected the first chamber, wherein the first chamber is configured for an IV fluid to flow from the IV fluid input connector to the IV fluid output connector;
an opening in the shared first chamber wall;
a differential pressure sensor in the second chamber configured to generate an electrical signal, wherein the differential pressure sensor is configured to be in fluidic communication with an IV fluid in the first chamber via the opening in the first chamber wall, wherein the differential pressure sensor is in fluidic communication with a reference fluid in the second chamber, and wherein the electrical signal is configured to be generated based at least on a difference in pressure between an IV fluid in the first chamber and the reference fluid in the second chamber; and
a reference tube with a first end and a second end, wherein the first end of the reference tube is fluidically connected to the second chamber, wherein the second end of the reference tube is connected to a membrane configured to be attached to a patient, and wherein the second chamber and the reference tube are filled with the reference fluid.

9. The system of claim 8, wherein the electrical signal is an analog signal.

10. The system of claim 8, wherein the electrical signal is a digital signal.

11. The system of claim 8, wherein the differential pressure sensor comprises an external membrane, wherein the external membrane is in fluid contact with IV fluid.

12. The system of claim 8, wherein the reference fluid and the IV fluid are the same fluid.

13. The system of claim 8, wherein the reference fluid and the IV fluid are not the same fluid.

14. The system of claim 8, wherein the first chamber and the second chamber are located inside a transducer housing, wherein an exterior wall of the transducer housing includes an identifier configured to be optically recognized, and wherein the identifier is associated with the reference fluid.

15. A method comprising:
electrically connecting a hemodynamic monitor to a differential pressure sensor of a transducer, wherein the transducer comprises:
a first chamber and a second chamber, wherein the first chamber and the second chamber share a first chamber wall that separates the first chamber and the second chamber;
a IV fluid output connector fluidically connected to the first chamber;
a IV fluid input connector connected the first chamber, wherein the first chamber is configured for an IV fluid to flow from the IV fluid input connector to the IV fluid output connector;
an opening in the shared first chamber wall;
a differential pressure sensor in the second chamber configured to generate an electrical signal, wherein the differential pressure sensor is configured to be in fluidic communication with an IV fluid in the first chamber via the opening in the first chamber wall, wherein the differential pressure sensor is in fluidic communication with a reference fluid in the second chamber, and wherein the electrical signal is configured to be generated based at least on a difference in pressure between an IV fluid in the first chamber and the reference fluid in the second chamber; and
a reference tube with a first end and a second end, wherein the first end of the reference tube is fluidically connected to the second chamber, wherein the second end of the reference tube is connected to a membrane configured to be attached to a patient, and wherein the second chamber and the reference tube are filled with the reference fluid;
connecting an IV fluid bag to the IV fluid input connector, wherein the IV fluid bag contains an IV fluid;
connecting a first end of a patient IV fluid tube to the IV fluid output connector;
connecting a second end of the patient IV fluid tube to a patient;
connecting the second end of the reference tube to the patient;
providing the IV fluid to the patient from the IV fluid bag via the transducer and the patient IV fluid tube;
generating the electrical signal with the differential pressure sensor;
transmitting the electrical signal to the hemodynamic monitor; and
measuring a blood pressure of the patient by the hemodynamic monitor based on at least the electrical signal.

16. The method of claim 15, wherein the electrical signal is an analog signal.

17. The method of claim 15, wherein the electrical signal is a digital signal.

18. The method of claim 15, wherein the differential pressure sensor comprises an external membrane, wherein the external membrane is in fluid contact with IV fluid.

19. The method of claim 15, wherein the reference fluid and the IV fluid are the same fluid.

20. The method of claim 15, wherein the reference fluid and the IV fluid are not the same fluid.
